(19) United States
(12) Patent Application Publication
(10) Pub. No.: US 2016/0022920 A1
(43) Pub. Date: Jan. 28, 2016

(54) Syringe

(71) Applicant: GAVIS PHARMACEUTICALS, LLC, Somerset, NJ (US)

(72) Inventor: Timothy Lloyd Reeves, Godfrey (CA)

(21) Appl. No.: 14/776,691

(22) PCT Filed: Mar. 14, 2014
(86) PCT No.: PCT/US14/28989

§ 371 Date: Sep. 14, 2015

Related U.S. Application Data
(60) Provisional application No. 61/785,499, filed on Mar. 14, 2013.

(51) Int. Cl. A61M 5/315; A61M 5/32

(52) U.S. Cl. CPC A61M 5/31563; A61M 5/32; A61M 2250/00

(57) ABSTRACT

A lock unit for a syringe provides a proper dose of an agent or medication from the syringe. The lock unit sets the dose of the syringe and is not thereafter affected by subsequent shipping or handling. The lock unit is applied to the plunger to limit the travel of the syringe piston in the syringe barrel thus limiting the dose that is delivered. Optionally, one or more syringes can be provided with a plurality of lock units of differing dimensions each set to deliver a specific dose of the agent when attached to a syringe.

### BACKGROUND OF THE INVENTION

1. Field of Invention

This invention relates to new syringes for the delivery of an agent, such as medications or nutritional supplements, to a human or veterinary patient. More particularly, the invention is directed to syringes that are designed to preclude errors in the delivery of the proper amount of agents, including medications or nutritional supplements. The syringe has a mechanism that controls the travel of the plunger of the syringe to assure the delivery of the proper amount of the agent.

2. Description of Related Art

Often different amounts of a substance or agent must be administered by a syringe depending on a number of factors. For example, not often do all human or veterinary patients receive identical doses of agents, such as active pharmaceuticals or drugs. Factors that influence a difference in drug dosage can include, for example, the size and weight of the patient, the age of the patient, and the sex of the patient. Currently, in order to administer the proper dose to the patient, drug delivery devices typically require that the device is manufactured such that the entire volume is metered to deliver the desired dose of the drug or, at the point of care, a caregiver must adjust the volume to be delivered. Both of these options, however, have certain inherent drawbacks.

In one case, the manufacturer is required to make several versions of a device, with each version including different volumes, usually incremental volumes, of the drug. Further, because several different versions of the device are required for different doses, both the distributor of the drug and the pharmacist are required to provide more “shelf space” for maintaining an inventory of the various devices containing different doses or amounts of the drug.

In the case where a caregiver adjusts the dosage, the caregiver might make an error in setting the proper dosage. This possibility is particularly a concern if the patient requires an immediate administration of a drug. Thus there remains a need for a delivery system that can be adjusted at a suitable dispensing location such as at a pharmacy or a nursing station.

In addition there are other uses for the syringe of the present invention. There are uses in analytical and diagnostic laboratories where doses of reagents are important. There are further uses in the agricultural area for the delivery to plants of nutrients and other substances.

A syringe directed to this problem of assuring the proper dose of a medication with the syringe is the subject of U.S. patent application Ser. No. 12/789,373, now abandoned. This syringe, if not properly used by the pharmacist in setting the dose, fails to achieve all of the desired benefits. The pharmacist needs to align colors on a ring dial on the plunger and on the syringe barrel and then lock the ring dial at the proper location. If the ring dial is not properly and fully locked, an error can result in the amount of medication delivered to the patient. This can be harmful to patients.

An object of one embodiment of the invention is to make the delivery of the proper amount of a medication essentially foolproof. One aspect is that one can easily determine by a cursory glance that a plunger locking mechanism has been properly set for the desired dosage of medication to be delivered to a patient. Another object of the present invention is to provide a device and method that reduce the possibility of errors by pharmacists or others in setting the dose to be dispensed by a syringe. An object is to use plunger locks with differing colors or other easily discerned indicia that render it easy to verify or “double check” the dose that a syringe is set to deliver. Each person in the medication delivery system can easily verify that the dose to be delivered is accurate.

### BRIEF DESCRIPTION OF THE INVENTION

The present invention comprises a syringe locking system wherein lock units surround a significant portion of the shaft of a syringe plunger. A desirable syringe plunger has a piston at a first end, a shaft attached to the piston, and a hand contact part at a second end. A syringe barrel surrounds a portion of the plunger and contains the medication or substance to be delivered to a patient. The lock unit of the locking system optionally attaches to the top of the barrel. The locking system wraps preferably around with the shaft of the plunger passing through the unit. The lock unit limits the travel of the plunger and thus the amount of medication that can be delivered by the syringe. By the use of lock units of varying heights, the travel of the syringe plunger can be limited and thus the medication dose set. One optional design is use of cylinder shaped locks. The lock units can be color or otherwise coded to assure that the proper locking unit is used on a syringe. These codes or indicia assist in preventing medication errors.

In one embodiment, the device also comprises a system that can be used by pharmacists or others in the preparation of syringes. In some uses of syringes, the syringes are provided to pharmacists in packs with a plurality of syringes which, when filled, provide kits or packets to the user or caregiver which desirably have two or more syringes. In such instances a packet has two syringes and a plurality of different lock units for each syringe. The length or dimension of the lock units is sized to provide a specific dose of an agent. The pharmacist selects and usually uses one appropriate lock unit of the same dimension for each syringe depending on the required dose. The pharmacist then disposes of the unused lock units or returns them to the source of the syringe and unit locks. In another aspect, when the user or caregiver receives the locked syringes, that person can see the applied lock unit, compare it to the prior used syringes, if available, and be assured that the dose in the present syringe is the same as previously used. This is a system and method of visually double checking the dose that is to be applied. Calculating the length or dimension of a lock unit to provide a specific dose of an agent from a specific syringe is within the skill of the art.

The invention includes a method of limiting a pre-filled syringe to delivery of a specific dose of an agent. The method has the step of filling a syringe with the agent. The syringe includes (1) a barrel and (2) a plunger. The plunger is slidable within the barrel, and the plunger includes a shaft extending from a piston to a plunger cap. The method then involves selected a lock unit by a dimension of the lock unit. The selected dimension correlates to a desired dose. Affixing the lock unit to a portion of the plunger, such as the shaft, cap, or other component, is then performed and is followed by limiting travel of the piston in the barrel by the affixed lock unit. Compressing the plunger in the barrel delivers a desired, pre-determined dose of the agent from the syringe.

### BRIEF DESCRIPTION OF THE DRAWING

FIG. 1 is an illustration of a lock unit and of a lock unit on the plunger of a syringe.

FIG. 2 is an illustration of a unit lock and of the barrel of a syringe with the fitting of the lock unit onto the barrel.

FIG. 3 is an illustration of a lock unit wherein the top part of the plunger fits into the unit lock to preclude reuse of the syringe.

FIG. 4 is an illustration of a unit lock wherein the top part of the plunger is held above the top surface of the lock unit so that the plunger can be gripped and removed from the syringe barrel for disposal of the syringe contents.

FIG. 5 is an illustration of a chosen lock unit on a syringe with the unused unit lock being discarded.

FIG. 6 is an illustration of the different size lock units for a syringe and for use in a syringe system.

FIG. 7 is an illustration of an optional improvement to the cap on the lower part of the syringe or the needle to make it easier for single hand removal of the cap.

FIG. 8 is an illustration of typical use of operations performed by the pharmacist in the filling of the syringes with a medication according to the present invention.

### DETAILED DESCRIPTION OF THE INVENTION

The invention will now be described below in its embodiments with reference to the appended drawings. The scope of the invention is not limited to this detailed description, or the drawings, but solely by the appended claims.

Often different amounts of a substance or agent must be administered depending on the situation. For example, not often do all human or veterinary patients receive identical doses of drugs. Factors that influence a difference in drug dosage can include, for example, the size and weight of the patient, the age of the patient, the sex of the patient, whether the patient is young or an adult, etc. Currently, in order to administer the proper dose to the patient, drug delivery devices typically require that the device is manufactured such that the entire volume is metered to deliver the desired dose of the drug or, at the point of care, a provider must adjust the volume to be delivered. Both of these options, however, have certain inherent drawbacks.

In one case, the manufacturer is required to make several versions of a device, with each version including different volumes, usually incremental volumes, of the drug. Further, because several different versions of the device are required for different doses, both the distributor of the drug and the pharmacist are required to provide more “shelf space” for maintaining an inventory of the various devices containing different amounts of the drug. The same type of problems holds true for substances such as substances for laboratory or agricultural use.

In the case where a provider adjusts the dosage, the provider might make an error in setting the proper dosage. This could be especially important if the patient requires an immediate administration of a drug. Thus there remains a need for a delivery system that can be adjusted at a suitable dispensing location such as a pharmacy, nursing station, or laboratory.

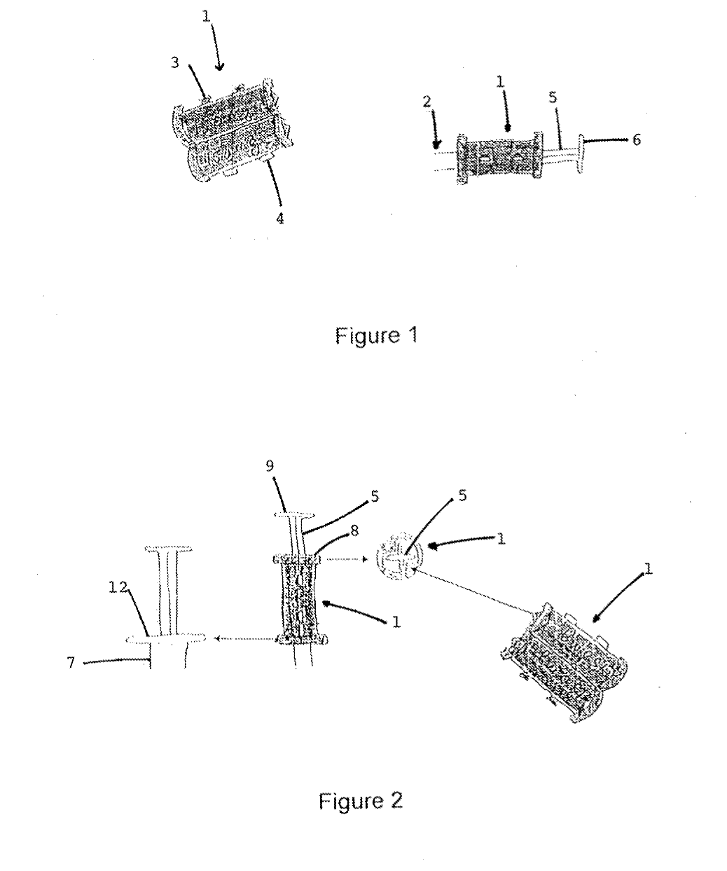

FIG. 1 is an illustration of the lock unit 1 for a syringe 2. The lock unit is in one aspect a cylinder hinged to open into two halves. The exposed edge of each hinged part has a lock, such as complementary male locking clip 3 and a female locking clip 4 to maintain the cylinder in a closed position when in use as a lock unit 1. In use, the cylinder is opened, fitted around the shaft 5 of a piston 6, closed, and held in a closed position via the lock or locking clips. This lock unit 1 limits the travel of the shaft 5 of the piston 6, thereby affecting the travel of the piston 6 in the syringe barrel, and thus the amount of an agent, such as a medication, delivered from the syringe.

Each dose guard or lock unit in the preferred embodiment includes a single “creased” piece of plastic with locking clips.” The pharmacist or other person selects the appropriate colored dose lock and “wraps” it around the syringe. The plastic “clips” prevent unlocking without significant effort on the part of the caregiver.

FIG. 2 is an illustration of the lock unit 1 fitted onto the shaft. 5 of a piston 6. The lock unit 1 has an upper or cap-receiving surface 8 with a structure to conform to the shape of the shaft 5 from the piston 6. In the illustration of FIG. 2, the piston shaft 5 has an “X” shape with the unit lock upper surface 8 having projections that fit into the “V” shaped grooves of the piston shaft 5. Other geometries and configurations are acceptable.

The base of the lock in the preferred embodiment surrounds the flange at the top of the syringe barrel. Triangular teeth in this embodiment at the top of the lock fit the “V-grooves” of the plunger. Ideally, the flange at the top of the barrel is round, to make fitting the teeth of the lock into the V-grooves of the plunger easier, regardless of the orientation of the plunger.

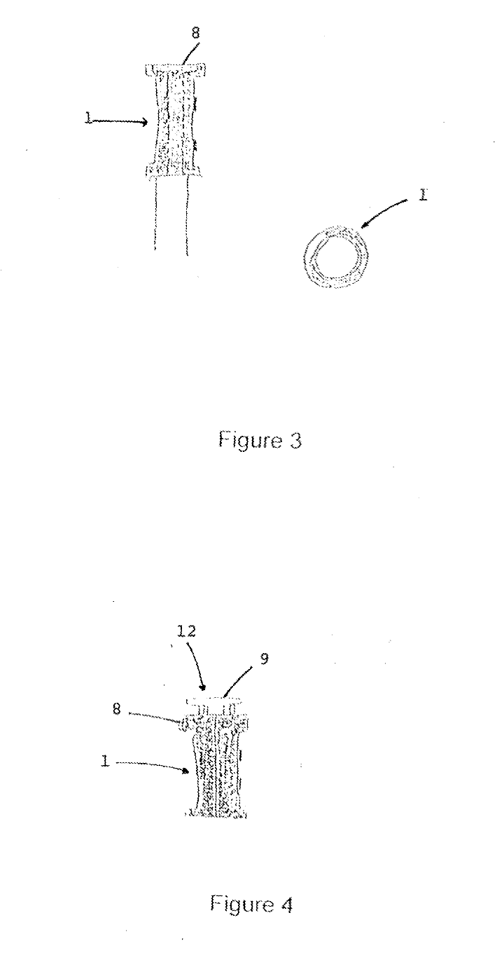

FIG. 3 shows the option of the cap 9 or top part of the piston shaft fitting into the top surface of the lock unit 8. In this way the piston shaft 5 and, consequently, the piston 6 are in a locked position. In this version the lock unit 1 allows the piston shaft 5 to move into the lock unit but not its removal from the lock unit.

The lock is a physical stop. The plunger in the preferred embodiment can be depressed to the top of the lock, and no further. This structure ensures that no more than the prescribed dose is administered. In this embodiment, the top of the lock is recessed so that the top flange of the plunger fits within the recess. This structure makes it much more difficult to pull the plunger out to try and re-use it.

FIG. 4 illustrates a version wherein the plunger shaft 5 and thus the piston 6 can be moved upward or out of the syringe barrel 7. The plunger 12 cannot be removed from the barrel 7 unless the lock unit 1 is detached from the top surface of the barrel or barrel flange 13. This can be achieved using a tool to open the lock unit 1. In this version the unit lock 1 has ribs projecting from the upper surface of the lock unit 1. This provides for a person to grip the plunger 12 top surface or cap 9 and pull it upward to raise or partially withdraw it in the syringe barrel 7.

While the recessed design in the preferred embodiment prevents re-use, this structure also prevents caregivers from washing the unused gel down the sink prior to disposal. A rib on the top of the lock, and no recess, can make it easier to grip and remove the plunger.

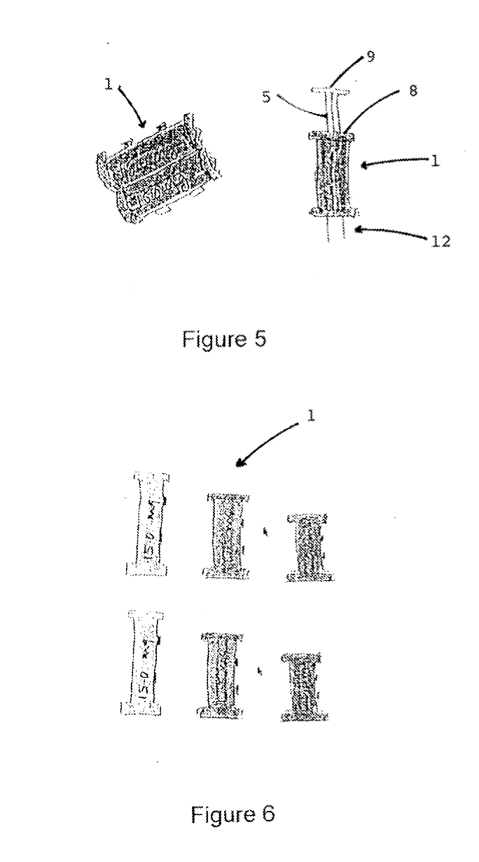

FIGS. 5 and 6 are directed to the preferred embodiment of syringe twin packs. In many instances syringes are provided to a pharmacist or others in twin packs from filling and forwarding to a caregiver. In another embodiment they are pre-filled, and the lock is applied by the pharmacist corresponding to a dose. With each syringe the pharmacist is provided with a plurality of lock units 1 of differing height dimensions, as shown in FIG. 6, for each syringe. FIG. 6 illustrates a twin pack of syringes having three lock units 1 for each syringe 2. Only one lock unit 1 is used with each syringe 2, and the others discarded or returned to the supplier. As discussed above, the height dimension of a lock unit determines the travel distance of the plunger 12 and thus the amount of medication delivered. As noted, if a syringe arrives at the caregiver with the lock unit open, it is a clear indication of a problem, and that syringe is not to be used.

Only the locked syringes in the preferred embodiment of the system and the method are returned to the twin pack. The unused locks are discarded. It is also desirable to label, such as with a sticker, that the dose has been set. If a caregiver receives a twin pack unlocked, the caregiver knows immediately that there is a problem.

Each twin pack in the preferred embodiment includes color coded dose guards, two for each possible dose, which is one for each syringe. Dose guards are desirably, clearly labeled with the dose value. No dose labels or gradations appear on the syringe in the preferred embodiment.

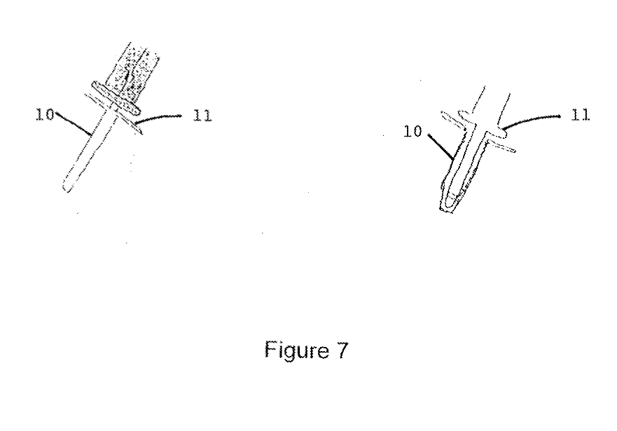

FIG. 7 illustrates an optional closure 10 for the lower part of a syringe 2. In this aspect, the closure 10 has a wider grip flange 11 to enhance one hand use of the syringe 2. This one hand in addition to holding the syringe 2 utilizes several fingers for removing the closure 10. Many times the other hand is in contact with the patient getting the patient ready to receive the medication.

The cap of the prior art is too difficult to remove. At a minimum, it requires two hands. This means no hand is available to stabilize an epileptic patient. An alternative design for the cap is to use a longer cap with a wide flange at the base of the barrel. The wider flange enables caregivers to dislodge the cap with one hand by wrapping their fingers around the barrel and pushing the cap flange away with their thumb.

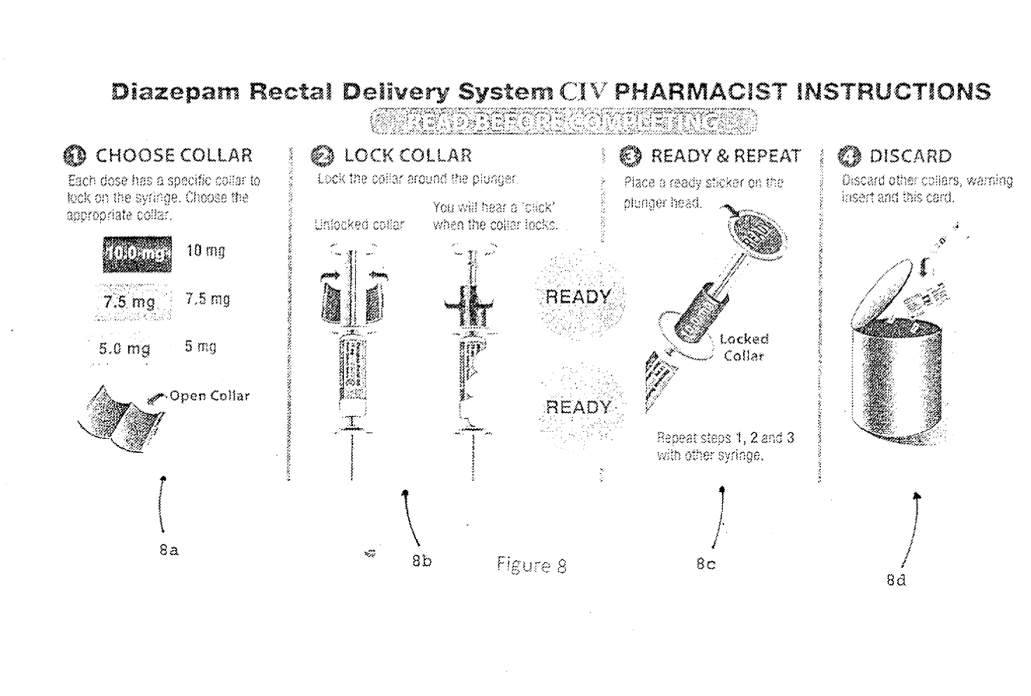

FIG. 8 illustrates a system and a method for using the invention. This system 14 for a syringe-type drug delivery system is easy to use by a pharmacist, caregiver, or other person. In one optional system, a collar locking unit 1 is positioned around the plunger 12 of the syringe 2 between the barrel 7 of the syringe and the end of the plunger as shown in FIG. 2. There can be several lock units of different lengths provided with the kit with different lengths corresponding to a particular volume of dose to be delivered as shown in FIG. 6. In use, the provider locks the appropriate size lock unit onto plunger 12 of the system 14 as required. The lock units are preferably of different colors, and a chart, preferably colored, is provided to instruct the pharmacist or other person on which lock unit 1 is to be used for a specific volume.

The method of the invention can include, as shown in FIG. 8a, selecting a lock unit such as collar. Each dose has a specific collar to lock on the syringe. Choosing the appropriate collar, as shown in FIG. 8b, sets the syringe for a specific dose of an agent. The selected lock unit or collar limits the compression or movement of the plunger within the syringe. This action of limiting the compression or movement can be accomplished with a variety of mechanical structures. Some of these structures can attach only to the plunger, such as its shaft, or to the syringe cylinder or barrel, such as its top surface or flange. Desirably, a “click” occurs when the collar locks. A label is desirably placed on the plunger head, as shown in FIG. 8c. These steps are repeated for other syringes in the kit. The other collars, warning insert, and instruction card are then discarded, as shown in FIG. 8d.

The locking unit mechanism is applicable to drug delivery devices suitable for subcutaneous, intramuscular, oral, buccal, otic, nasal, topical, or rectal use in humans and veterinary patients, or for delivery medium in agricultural use.

In one desirable embodiment, the syringe contains medication for rectal use. In a more desirable embodiment, the syringe contains Diazepam rectal gel. In yet another embodiment, the type of the syringe is made of plastic, a metallic needle, with a cap enclosing the needle, as shown in FIG. 7 for one optional type of cap. The pharmacist optionally obtains one or two syringes with Diazepam rectal gel that is provided as a kit with several locking units for each syringe. The locking units have different lengths and preferably with each length color coded. The syringes and locking units are preferably supplied with an instructional sheet.

In another embodiment, the locking unit doses with a click to notify the provider that it is locked. In yet another embodiment the collar unit, once locked, cannot be unlocked, as shown in FIG. 1.

Syringe kits of this invention surprisingly yield better results than prior art kits. The following comparative Example 1 shows the method of use of this invention and the results from a kit as described in U.S. patent application Ser. No. 12/789,373, incorporated herein by reference, with the results of the kit illustrated in FIG. 8. The results from the current invention are as shown in the Table accompanying Example 1. As can be seen, the present invention gives surprisingly better results than the prior art design. The pharmacists were able to set the correct dosage more consistently and lock the unit with the present invention. Accurate dosage to a patient is important as is the ability of pharmacists to set the correct dosage consistently and lock the plunger to deliver the proper dose.

### Example and Comparative Example

A study was conducted following U.S. application Ser. No. 12/789,373 in which 18 pharmacists were given two kits of Diazepam Rectal Gel, one in 10 mg strength and one in 20 mg strength, for a total of eight syringes per pharmacist. Three pharmacists in this study did not lock the syringe. One pharmacist left one syringe unlocked. A second pharmacist left five syringes unlocked, and a third pharmacist left seven syringes unlocked.

The results of a kit as described for this invention and FIG. 8, with 15 pharmacists each given two Diazepam Rectal Gel kits in 10 mg and 20 mg strengths for a total of eight syringes per pharmacist, is as follows:

| Pharmacist | | | |
|---|---|---|---|
| Task | Success | Success w/difficulty | Failures |
| Select correct | 14 | 0 | Pharmacist # 5 |
| dose collar | | | set dose wrong |
| | | | on two syringes. |
| | | | Likely mis-reading |
| | | | of script. |
| Lock collar on | 11 | 4 pharmacists | None |
| plunger shaft | | One or more clips | |
| | | broke per pharmacist; | |
| | | appeared to be the | |
| | | result of manufacturing | |
| | | defect. | |

Thus, the present invention was found to be superior.

The invention can have additional optional features. For example, the collar can consist of a single “creased” piece of plastic with locking clips, as shown in FIG. 1. The pharmacist selects the appropriate colored dose collar and “wraps” it around the syringe. The locking clips are optionally designed such that they prevent locking or unlocking without significant effort. They are not necessarily of cylinder shape but can be of other shapes with different overall height.

Additionally, an optional design feature is for the base of the collar to surround the top of the barrel end and thus hold the collar at the top of the barrel. Another feature is for teeth at the top of collar to fit into optional V-grooves of the plunger. This prevents accidentally pulling the plunger shall out of the syringe. Preferably, the flame at the barrel top would be round so that it is easier to lock the collar without having to adjust the orientation of the plunger shaft with regards to the barrel, as shown in FIG. 2.

Optionally, the top of the collar has a recess that matches and locks in with the flange of the plunger, thus irreversibly locking upon delivering the substance to the patient, as shown in FIG. 3.

1. A syringe comprising:
a barrel;
a plunger, the plunger is slideable within the barrel, and the plunger includes a shaft extending from a piston to a plunger cap;
a lock unit affixable to the plunger, the lock unit is dimensioned to limit travel of the piston on the plunger in the barrel to deliver a dose of an agent from the syringe; and
wherein the lock unit comprises a first surface, the first surface includes a recess of a dimension selected to receive a piston cap within the recess.

2. (canceled)

3. The syringe as in claim 1 wherein the lock unit comprises two parts connected on one edge and adapted to be fastened on the other edge.

4. The syringe as in claim 1 wherein the lock unit attaches to a flange at a first end of the barrel.

5. The syringe as in claim 4 wherein the flange is circular.

6. (canceled)

7. The syringe as in claim 1 wherein the lock unit has a first surface, at least one rib projecting upward from the first surface, the at least one rib contacts a cap of said piston to enable gripping and removing the piston from the barrel.

8. The syringe as in claim 1 wherein each of a plurality of lock units of differing dimensions from a first surface to a second surface are selectable to limit the travel of the piston in the barrel to dispense different amounts of an agent.

9. A syringe system comprising:
a plurality of syringes, each of the syringes includes:
(i) a barrel;
(ii) a plunger, the plunger is slideable within the barrel, and the plunger includes a shaft extending from a piston to a plunger cap;
(iii) a plurality of lock units, each lock unit affixable to the plunger and dimensioned to limit travel of the piston on the plunger in the barrel to deliver a specific dose of an agent from the syringe; and
whereby selecting the lock unit for one syringe is determined by a desired dose of the agent to be delivered by the syringe;
wherein the lock unit comprise a first surface, the first surface includes a recess of a dimension selected to receive a piston cap within the recess.

10. The syringe system as in claim 9 wherein the plurality of syringes includes at least two syringes, each of the plurality of syringes is associated with at least two lock units, each lock unit having a differing dimension from a first surface to a second surface, whereby selecting and affixing lock units of equal dimension for each syringe limits the travel of the piston in the barrel of each syringe to dispense an equal dose of the agent from each syringe.

11. The syringe system as in claim 10 wherein there are two syringes with three lock units of differing dimensions associated with each syringe, each of the two syringes is filled with the same agent, each filled syringe when attached to a lock unit of the same dimension delivers the same dose of the agent.

12. The syringe system as in claim 9 comprising a kit containing the plurality of syringes and the plurality of locking units, wherein locking units of one color correspond to a common length and a particular volume of the agent to be dispensed.

13. The syringe of claim 1 wherein the barrel is pre-filled with diazepam rectal gel.

14. The syringe of claim 1 which is adapted for human use.

15. The syringe of claim 1 which is adapted for veterinary use.

16. The syringe of claim 1 which is adapted for agricultural use.

17. A method for limiting a pre-filled syringe to provide a specific dose comprising:
filling a syringe with an agent, the syringe includes:
(i) a barrel; and
(ii) a plunger, the plunger is slideable within the barrel, and the plunger includes a shaft extending from a piston to a plunger cap;
selecting a lock unit by a dimension of the lock unit, the selected dimension correlating to a desired dose;
affixing the lock unit to the plunger;
limiting travel of the piston in the barrel by the affixed lock unit to deliver a dose of the agent from the syringe; and
wherein the lock unit comprises a first surface, the first surface includes a recess of a dimension selected to receive a piston cap within the recess.

18. The method of claim 17 wherein the agent is diazepam and the specific dose of diazepam is administered as a rectal gel to treat seizures.

19. The syringe as in claim 1 wherein the lock unit comprises an upper surface, the upper surface includes a projection that fits into “V” shaped grooves of the piston shaft.

20. The syringe as in claim 19 wherein the projection is in the form of triangular teeth.

21. The syringe as in claim 3 wherein fastening of the two edges creates a click sound.

22. The syringe as in claim 1 wherein the syringe comprises a cap at a base of the barrel, the cap includes a wide flange.
